(19) United States
(12) Patent Application Publication
(10) Pub. No.: US 2005/0096657 A1
(43) Pub. Date: May 5, 2005

(54) Osteosynthesis or arthrodesis material comprising a bony plate

(76) Inventors: Alex Autericque, Idron (FR); Jean-Claude Huot, Pau (FR); Yves Adam, Authie (FR); Cornelius Cioloca, Villejuif (FR); Bernard Villaret, Croix-Chapeau (FR)

Correspondence Address: YOUNG & THOMPSON, 745 SOUTH 23RD STREET, 2ND FLOOR, ARLINGTON, VA, 22202, US

(21) Appl. No.: 10/925,960

(22) Filed: Aug. 26, 2004

Related U.S. Application Data
(63) Continuation-in-part of application No. PCT/FR03/00620, filed on Feb. 26, 2003.

(30) Foreign Application Priority Data
Feb. 26, 2002 (FR) 02 02422

(51) Int. Cl. A61F 2/30

(52) U.S. Cl. 606/69

(57) ABSTRACT

The invention relates to a plate (2) with a slot (7), adjacent to at least one hole (4, 6) therein, which traverses the whole of said plate and which extends along said hole (4, 6) on a part of the perimeter of said hole (4, 6). The hole (4, 6) and slot (7) thus define a band of material (8) between them which is flexibly elastic. The band (8) comprises locking means (9) on the edge thereof defining the hole (4, 6), which project towards the interior of the hole (4, 6). The object (3), for placement in said hole (4, 6), comprises complementary locking means (10) which, when said object (3) is engaged in said hole (4, 6) come into engagement with the locking means (9) on the band of material (8) such as to connect said object (3) to the plate (2) or to fix said plate (2) to a bone.

The present invention relates to osteosynthesis or arthrodesis material comprising a bony plate. This material may notably be an arthrodesis material of cervical vertebrae, implanted by anterior route.

It is well known to use rigid plates for repairing or consolidating a bone, or for realising an arthrodesis, this plate comprising holes for letting through screws enabling the attachment thereof to the bone. It is also known to assemble a support structure of an intervertebral graft, such as an intervertebral cage, to a bony plate.

The efficiency of an osteosynthesis or of an arthrodesis implies perfect immobilisation of the bones or portions of treated bones, and the shortcoming of the existing materials is that they are not totally satisfactory from this point of view. Indeed, the stresses exerted on the material by the bone or portions of bones may lead to slight loosening of the screws, to the extent that the plate may show a slight clearance with respect to these bones or portions of bone. This loosening occurs in particular when the bone(s) are more or less brittle, or when the anatomy of this bone or these bones is such that they do not provide vast possibilities of insertion of the screws, as it is particularly the case for an arthrodesis of cervical vertebrae.

The assembly of support structure of an intervertebral graft to a bony plate may also not prove totally satisfactory with the existing materials.

The purpose of the present invention is to remedy these essential shortcomings.

The osteosynthesis or arthrodesis material affected comprises, in a manner known in itself, a bony plate and at least one object intended to be connected to this plate or for attaching this plate to a bone.

In this view, in the material in question,
the plate includes, by at least one hole comprised therein, a through slot which extends along said hole over a portion of the perimeter of said hole, whereas said hole and said slot thus delineate together a band of material which is flexibly elastic; and
the object intended for placement in this hole comprises locking means liable to come into engagement, when said object is engaged in the hole, with said band of material, in order to connect said object to the plate or to attach this plate to a bone, said flexible elasticity being such that the band normally have a position directed towards the interior of the hole, in order to come into engagement with said locking means, but that the band may be deformed elastically towards the exterior of this hole, so as to allow the engagement of said object into the hole by rotation.

According to one embodiment, the band comprises a smooth face on its face delineating the hole and the object comprises a facetted portion, for example a hexagonal portion, whereas the facets are consecutive and two consecutive facets delineates together a ridge; the ridges, in angular positions of the object, bear against the band to allow said object to pivot and, in other angular positions of the object, each facet may abut against this smooth face of the band, the elastic recall of this band thus locking the object.

According to another embodiment, the band comprises, on the edge thereof delineating the hole, complementary locking means which project towards the interior of the hole, which complementary locking means come into engagement with the locking means of said object. The band can be deformed elastically towards the exterior of the hole, over a distance at least equal to the height of the locking means.

Said complementary locking means included in the band may be formed by a series of notches and said object may be a screw for fixing the plate to a bone, this screw comprising a series of notches forming said locking means included in the object.

Said complementary locking means may be formed by at least one locking toe and said object may be a setting prop and/or a support structure of an intervertebral graft such as an intervertebral cage, this setting prop and/or this support structure comprising reception cavities of said locking toes, these cavities forming said locking means included in the object.

The hole aforementioned of the plate may be circular or oblong. In the latter case, said slot extends along one of the elongated edges delineating longitudinally the hole, as well as possibly along a portion of at least one of the curved edges delineating the ends of the hole, if it is necessary to obtain the flexible elasticity requested.

The axis of the hole aforementioned may be provided along an axis perpendicular to the plate or non perpendicular to this plate. In both cases, the locking means included in the plate have faces arranged parallel to the axis of the hole and the locking means included in the object have faces arranged parallel to the axis of the object, to enable the locking means included in the object to come into co-operation with the locking means included in the plate when the object is engaged in the hole.

According to a particularly interesting use of the invention, the plate is intended to be used for realising a vertebral arthrodesis, notably a cervical arthrodesis with implantation by anterior route, first of all.

This plate may be ‘X’-shaped and include four holes provided in its branches.

The central portion of the plate is intended to be placed opposite the intervertebral space, and the holes of the branches are intended to receive the fixing screw of the plate against the anterior face of the vertebral bodies.

A hole or the holes situated at at least one longitudinal end of the plate may be oblong. In this case, this or these holes are advantageously orientated so that their longitudinal axis are parallel to the longitudinal axis of the plate. This orientation of the hole(s), together with the facet(s) situated proximate the smooth face(s) of the band(s), allows the screw(s) to slightly slide within the hole(s) so as to allow an adjustment of the position of the screw(s) with respect to the plate.

The plate may be formed, as seen laterally, into a chevron, in order to reconstruct anatomic lordosis of the vertebrae.

For better understanding, the invention is again described below with reference to the appended schematic drawing representing, by way of non limiting example, two possible embodiments of the material in question.

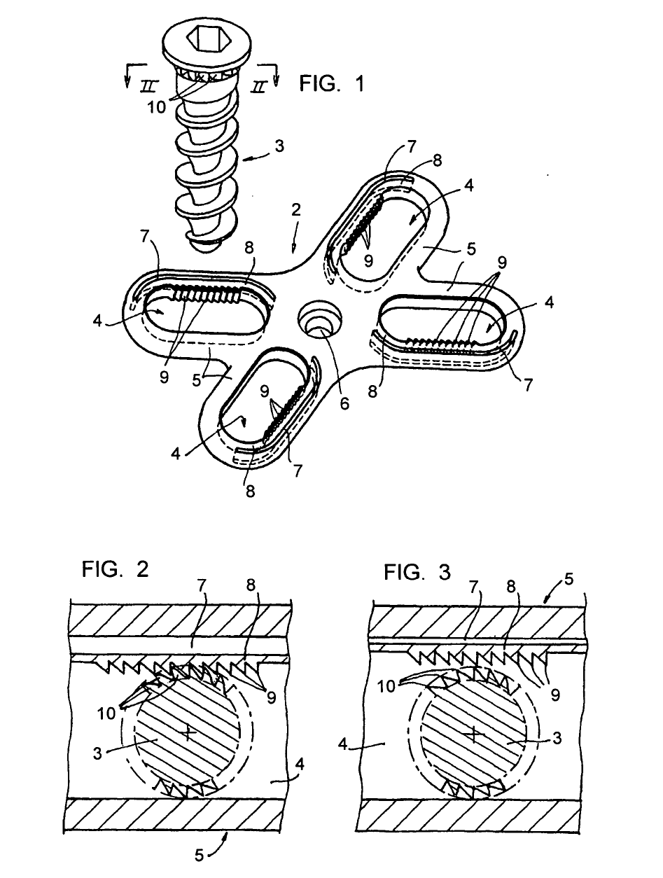

FIG. 1 is a perspective view of a plate and of a screw included in this material, according to a first embodiment;

FIG. 2 is a sectional view of a screw along the line II-II of FIG. 1, after engagement in a hole included in the plate to accommodate this screw, in an angular position of the screw;

FIG. 3 is a view similar to FIG. 2, in another angular position of the screw;

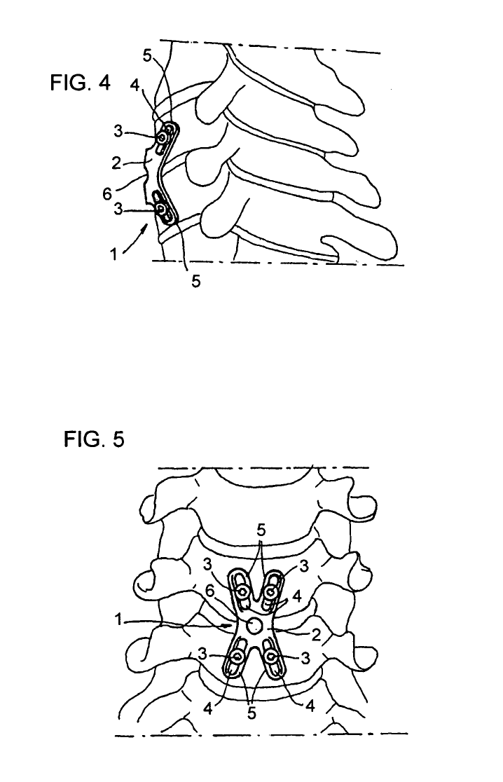

FIG. 4 is a side view of cervical vertebrae, on two of which the material is placed;

FIG. 5 is a front view of these cervical vertebrae and of this material;

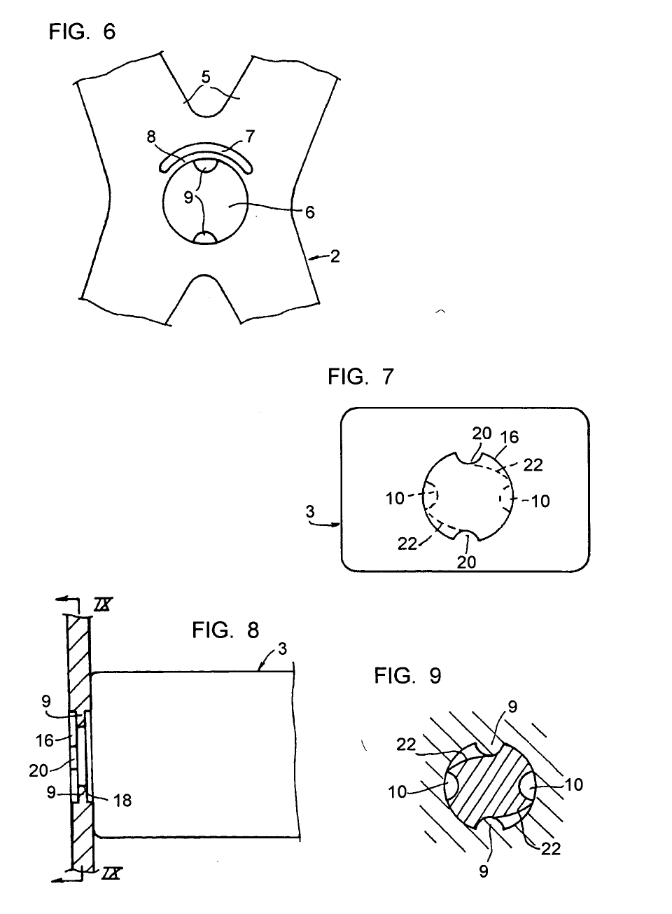

FIG. 6 is a partial view of the plate according to the second embodiment;

FIG. 7 is a front view of an intervertebral cage intended to be interconnected with this plate;

FIG. 8 is a side view of the plate and of this cage assembled to one another, the plate being seen as a medial longitudinal section, and

FIG. 9 is a sectional view of a stud included in the cage for assembly thereof to the plate, along the line IX-IX of FIG. 8.

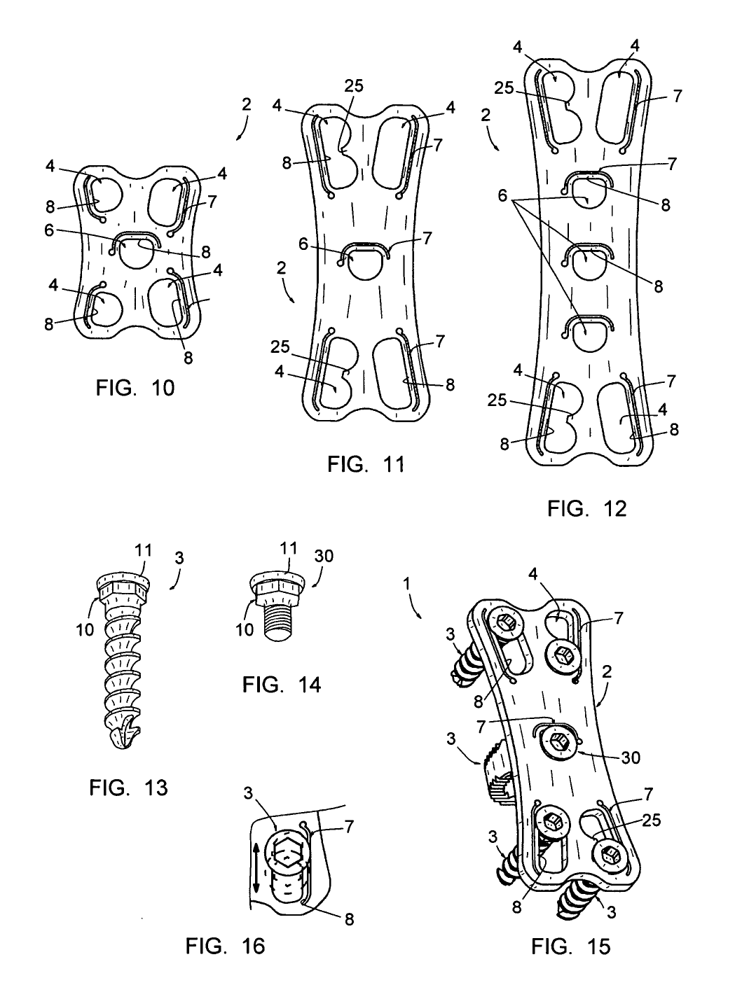

FIGS. 10 to 12 are planar views of three plates according to another embodiment;

FIGS. 13 and 14 are perspective views respectively of a screw intended to engage into one of the lateral holes of a plate shown on FIGS. 10 to 12 and of a screw intended to engage into a central hole included in each of these plates;

FIG. 15 is a perspective view of the plate shown on FIG. 11 after placement of screws shown on FIG. 13 and, for assembly of an intervertebral cage, after placement of a screw shown by FIG. 14, and

FIG. 16 is an enlarged view of a further embodiment.

For simplification purposes, the elements or portions of elements appearing as identical or similar from one embodiment to the next, are designated by the same digital references.

FIGS. 1, 4 and 5 represent a cervical vertebral arthrodesis material 1, comprising a plate 2 and screw 3 for fixing this plate to cervical vertebrae, the material 1 being intended for placement by anterior route.

The plate 2 is “X”-shaped and includes four oblong holes 4 provided the branches 5 thereof, parallel to the longitudinal axis of each of these branches 5, to accommodate the screw 3. As it appears on FIGS. 4 and 5, it is sized in order to extend along the anterior faces of two consecutive vertebral bodies and is formed, as seen laterally, into a chevron, in order to reconstruct the anatomic lordosis of the vertebrae to which it is fixed.

The plate 2 also comprise a central countersunk hole 6 enabling assembly thereof on a setting prop and/or the assembly of a support structure of an intervertebral graft, such as an intervertebral cage. As shown by FIGS. 4 and 5, this hole 6 is intended for placement at the intervertebral space of both vertebrae treated.

The plate 2 is made of titanium or of titanium alloy of type currently used to produce osteosynthesis or arthrodesis material, in particular the so-called “T40”-titanium or the so-called “TA6V”-titanium alloy.

As it appears on the Figures, each branch 5 possesses a through slot 7 which extends along one of the elongated edges delineating longitudinally the hole 4 as well as along portions of curved edges delineating the ends of this hole 4. The latter and this slot 7 thereby delineate together a band of material 8 which is flexibly elastic, this band 8 comprising, on its edge delineating the hole 4, notches 9 which project towards the interior of the hole 4.

The width of the slot 7 and said flexible elasticity are such that these notches 9 normally adopt a position directed towards the interior of the hole 4, but that the band 8 may be deformed elastically towards the exterior of this hole 4, over a distance at least equal to the height of the notches 9.

As shown more particularly on FIGS. 2 and 3, each notch 9 has a face oriented slantwise with respect to the longitudinal direction of the band 8 and a face oriented substantially perpendicular to the band 8, said faces oriented slantwise being anterior with respect to said faces oriented substantially perpendicular, in the tightening direction of the corresponding screw 3.

Each screw 3 may be tightened clockwise and shows a series of notches 10 provided below its head 11. As shown on FIGS. 2 and 3, each notch 10 has a face oriented slantwise with respect to the radial direction of the screw 3 and a face oriented substantially in the radial direction of the screw 3, said faces oriented slantwise being anterior with respect to said faces oriented substantially in the radial direction, in the tightening direction of the screw 3.

As can be understood with reference to FIGS. 2 and 3, each screw 3 is engaged through the corresponding hole 4 and is screwed in the bone until its notches 10 are situated at the notches 9 of the plate 2; the screwing may be continued until complete tightening of the screw 3, the notches 9 of the plate 2 clearing before the notches 10 of the screw, thanks to said flexible elasticity of the band 8; when the complete tightening position of the screw 3 has been reached, the notches 9 come into engagement with the notches 10 and prevent the screw 3 from getting loose with respect to the plate 2.

FIG. 6 represents a plate 2 whereof the hole 6 is surrounded by a slot 7 which delineates a band 8. The band 8 and the plate 2 comprise two locking toes 9, arranged diametrically opposite one another.

It appears on FIG. 8 that the thickness of these toes 9 is smaller than that of the plate 2 and they are recessed from the main faces of this plate 2.

The hole 6 is intended to receive an intervertebral cage 3, visible on FIGS. 7 and 8, for receiving a graft intended for bony fusion of the vertebrae.

As appears on FIGS. 7 to 9, this cage 3 comprises an assembly stud exhibiting a groove delineated by a proximal neck 16 and a distal neck 17.

The proximal neck 16 possesses two nicks 20 liable to be engaged on the toes 9 and is of such thickness that it may come into engagement behind these toes 9 when the distal neck 17, which is deprived of nicks, abuts against the toes 9 and the cage 3 is pivoted around its longitudinal axis.

The zone 18 of the stud situated between the necks 16, 17 comprises two nicks superimposed on the nicks 20 of the neck 16, two housings 10 accommodating toes 9 and two ramps 22 separating these nicks from these housings 10.

As can be understood with reference to the drawing, from the insertion position of said stud in the hole 6, the cage 3 may be pivoted around 90°, which leads the ramps 22 to push the toe 9 integral with the band 8 until the housings 10 face the toes 9. The elastic recall of the band 8 then brings the toe 9 integral with the band 8 into the corresponding nick, which ensures locking of the cage 3 with respect to the plate 2.

The hole 6 of the plate 2 according this second embodiment may obviously accommodate a screw 3 fitted with notches 11 having shapes corresponding to that of the toes 9.

FIGS. 10 to 12 show three plates 2 of different length according to another embodiment. These plates 2 show tapered portions and comprise the holes 4 at their lateral zones, which are widened. According to their sizes, they may present one or several holes 6, notably three holes 6 as shown on FIG. 12, to enable placement of one or several intervertebral implants 3.

Each plate 2 comprises a slot 7 along each hole 4 and 6, forming a band 8 whereof the central portion is rectilinear. This band 8 shows a smooth face on its side delineating the hole 4 or 6, this smooth face forming a locking means for the screws 3 or 30 represented on FIGS. 13 and 14, as described below.

According to the size of a plate 2, the holes may be adjusted to the screws 3 and 30, as is the case of the holes 4 situated on the left of the plate shown by FIG. 10, or may be more or less elongated, as is the case of the holes 4 situated on the right of the plate shown by FIGS. 10 to 12. Certain holes 4 may have a medial protrusion 25 on their edge opposite the band 8, delineating two housings adjusted to a screw 3, these housings thus delineating two accurate positions for these screws 3 (see holes 4 situated on the left of FIGS. 11 and 12).

The plates 2 represented on FIGS. 10 to 12 are part of a set of plates of different sizes.

The screws 3 and 30 each comprise a hexagonal facetted portion 10, said facets being consecutive and two consecutive facets delineating together a ridge. These ridges, in angular positions of these screws 3, 30, bear against the band 8 to allow said screws to pivot in the holes 4 and 6 and, in other angular positions of these screws 3, 30, each facet may abut against this smooth face of the band 8, the elastic recall of this band 8 thus locking the screws 3, 30.

The screw 30 comprises a thread enabling said screw to be tightened in a tapered bore included in the intervertebral implant 3 and thus enables assembly of this implant 3 on a plate 2 by screwing, as shown on FIG. 15.

According to a further embodiment shown on FIG. 16, the holes 4 are orientated so that their longitudinal axis are parallel to the longitudinal axis of the plate. This orientation of the hole(s) 4, together with the facets situated proximate the smooth faces of the bands 8, allows the screws 3 to slightly slide within the holes 4 so as to allow an adjustment of the position of the screws 3 with respect to the plate 2.

It appears from the foregoing that the invention brings a decisive improvement to the previous art, by providing an osteosynthesis or arthrodesis material enabling perfect immobilisation of the bones or of portions of bones treated. Indeed, this material ensures perfect hold of the complete tightening of the screws even when the bone(s) undergo repeated loads and/or the bone(s) are more or less brittle, and/or the anatomy of the bone(s) treated only enables reduced insertion depth of the screw in this bone or these bones, and enables perfect assembly of a cage 3 to the plate 2.

It goes without saying that the invention is not limited to the embodiment described above with exemplification purposes, but conversely includes all the embodiment variations covered by the appended claims. Thus, one or several holes 4 may be circular, and one or several holes 4 may be provided according an axis non perpendicular to this plate; the faces aforementioned of the notches 9 only have faces arranged parallel to the axis of the hole 4 and the faces aforementioned of the notches 10 remain arranged parallel to the axis of the screw 3, to enable the notches 10 to come into co-operation with the notches 9 when the screw 3 is engaged in the hole 4.

1. An osteosynthesis or arthrodesis material comprising a bony plate and at least one object intended to be connected to this plate or for attaching this plate to a bone, wherein:
the plate includes, by at least one hole comprised therein, a through slot which extends along said hole over a portion of the perimeter of said hole, whereas said hole and said slot thus delineate together a band of material which is flexibly elastic; and
the object intended for placement in this hole comprises locking means liable to come into engagement, when said object is engaged in the hole, with said band of material, in order to connect said object to the plate or to attach this plate to a bone, said flexible elasticity being such that the band normally have a position directed towards the interior of the hole, in order to come into engagement with said locking means, but that the band may be deformed elastically towards the exterior of this hole, so as to allow the engagement of said object into the hole by rotation.

2. Osteosynthesis or arthrodesis material according to claim 1, wherein the band comprises a smooth face on its face delineating the hole and the object comprises a facetted portion, whereas the facets are consecutive and two consecutive facets delineates together a ridge; the ridges, in angular positions of the object, bear against the band to allow said object to pivot and, in other angular positions of the object, each facet may abut against this smooth face of the band, the elastic recall of this band thus locking the object.

3. Osteosynthesis or arthrodesis material according to claim 1, wherein the band comprises, on the edge thereof delineating the hole, complementary locking means which project towards the interior of the hole, which complementary locking means come into engagement with the locking means of said object.

4. Osteosynthesis or arthrodesis material according to claim 3, wherein said complementary locking means included in the band are formed by a series of notches and said object may be a screw for fixing the plate to a bone, this screw comprising a series of notches forming said locking means included in the object.

5. Osteosynthesis or arthrodesis material according to claim 3, wherein said complementary locking means are formed by at least one locking toe and said object is a setting prop and/or a support structure of an intervertebral graft such as an intervertebral cage, this setting prop and/or this support structure comprising reception cavities of said locking toes, these cavities forming said locking means included in the object.

6. Osteosynthesis or arthrodesis material according to claim 1, wherein said hole is oblong, said slot extends along one of the elongated edges delineating longitudinally the hole, as well as possibly along a portion of at least one of the curved edges delineating the ends of the hole.

7. Osteosynthesis or arthrodesis material according to claim 3, wherein said hole and said object have axis, the axis of the hole being non perpendicular to the plate and said complementary locking means having faces arranged parallel to said axis of the hole, and the locking means included in the object having faces arranged parallel to the axis of the object, to enable the locking means included in the object to come into co-operation with the complementary locking means included in the plate when the object is engaged in the hole.

8. Osteosynthesis or arthrodesis material according to claim 1, wherein the plate is ‘X’-shaped and include four holes provided in its branches.

9. Osteosynthesis or arthrodesis material according to claim 1, wherein a hole or the holes situated at at least one longitudinal end of the plate are oblong, this or these holes being orientated so that their longitudinal axis are parallel to the longitudinal axis of the plate.

10. Osteosynthesis or arthrodesis material according to claim 1, wherein the plate is formed, as seen laterally, into a chevron.
